(19) United States
(12) Patent Application Publication
(10) Pub. No.: US 2013/0224771 A1
(43) Pub. Date: Aug. 29, 2013

(54) ANTI-MULLERIAN HORMONE DETECTION IN WHOLE BLOOD

(71) Applicants: Thomas W. McDade, Evanston, IL (US); William E. Funk, Evanston, IL (US); David B. Seifer, Holmdel, NJ (US)

(72) Inventors: Thomas W. McDade, Evanston, IL (US); William E. Funk, Evanston, IL (US); David B. Seifer, Holmdel, NJ (US)

(73) Assignee: Northwestern University, Evanston, IL (US)

(21) Appl. No.: 13/772,796

(22) Filed: Feb. 21, 2013

Related U.S. Application Data
(60) Provisional application No. 61/601,195, filed on Feb. 21, 2012.

(51) Int. Cl. G01N 33/74

(52) U.S. Cl. CPC G01N 33/74
USPC 435/7.92; 435/7.1; 435/287.2

(57) ABSTRACT

The present invention provides methods, kits, compositions, and devices for detecting Anti-Mullerian hormone (AMH) in whole blood samples. In certain embodiments, the methods, kits, compositions, and devices employ immunoassays that generate a colorimetric or fluorescent signal (e.g., using antibodies conjugated to gold nanoparticles or fluorescent particles) where the signal generated is proportional to the approximate concentration of AMH in a whole blood sample. In particular embodiments, the present invention provides quantitative or semi-quantitative lateral flow immunoassay devices and kits for detecting AMH at home (e.g., in order for women to estimate their ovarian age or diagnose polycystic ovarian syndrome).

The present application claims priority to U.S. Provisional Application Serial Number 61/601,195 filed Feb. 21, 2012, which is herein incorporated by reference in its entirety.

### FIELD OF THE INVENTION

The present invention provides methods, kits, compositions, and devices for detecting Anti-Mullerian hormone (AMH) in whole blood samples. In certain embodiments, the methods, kits, compositions, and devices employ immunoassays that generate a colorimetric signal (e.g., using antibodies conjugated to gold nanoparticles or fluorescent particles) where the signal generated is proportional to the approximate concentration of AMH in a whole blood sample. In particular embodiments, the present invention provides quantitative or semi-quantitative lateral flow immunoassay devices and kits for detecting AMH at home or at the point of care (e.g., in order for women to estimate their ovarian age or diagnose polycystic ovarian syndrome).

### BACKGROUND

Anti-Müllerian hormone (AMH), also called Müllerian inhibiting substance, has recently emerged as an important biomarker of ovarian reserve that has the potential to facilitate new research on fecundability, infertility, and reproductive aging. AMH is a glycoprotein dimer produced by granulosa cells from small pre-antral and antral follicles in the ovary, and it is hypothesized to inhibit the recruitment of primordial follicles into the pool of growing follicles (Durlinger, et al., 2002). Prior studies suggest that AMH represents a useful measure of ovarian reserve since it plays an important role in early stage follicle development, can be quantified in serum or plasma, and because levels fluctuate minimally during the menstrual cycle (Fauser, et al., 2002, Somunkiran, et al., 2007). Concentrations show a progressive decrease with age (Lee, et al., 1996, Seifer, et al., 2011, Lie Fong, et al, 2012) and predict timing of menopause in advance of clinical symptoms (e.g., menstrual irregularity) (Freeman, et al, 2012, Kaori et al, 2012, Tehrani, et al., 2009, 2013, van Rooij, et al., 2004). Moreover, reduced AMH has been associated with lower fecundability in small population-based studies (Steiner, et al., 2011), and among women undergoing ovarian stimulation for in vitro fertilization, AMH predicts ovarian response and pregnancy (Seifer et al, 2002, van Rooij, Tonkelaar, Broekmans, Looman, Scheffer, de Jong, Themmen and to Velde, 2004, McIlveen, et al., 2007, Kwee, et al., 2008, van Rooij, et al., 2002, Satwik et al, 2012). More recently, AMH values have been used to assess the impact of cancer treatments on ovarian reserve in female cancer survivors (Dillon et al, 2012, Fong, et al., 2008, Keizer-Schrama, et al., 2007). An obstacle to the measurement of AMH in community-based studies, or studies requiring multiple blood draws, is the requirement for venous blood. Venipuncture blood draws are costly, invasive, and must be performed by a trained phlebotomist in close proximity to a facility where blood samples can be centrifuged, separated (to generate plasma or serum), and frozen. What is needed are methods and devices that do not rely on venous blood and that can be employed with whole blood. In addition, the concentration of AMH is sensitive to how venipuncture blood samples are handled and stored, and concentrations may increase or decrease as a result of sample handling. What is needed are methods and devices that do not rely on venous blood and that can be employed with whole blood (e.g., collected from a finger stick).

### SUMMARY OF THE INVENTION

The present invention provides methods, kits, compositions, and devices for detecting Anti-Mullerian hormone (AMH) in whole blood samples (e.g., capillary blood samples). In certain embodiments, the methods, kits, compositions, and devices employ immunoassays that generate a colorimetric signal (e.g., using antibodies conjugated to gold nanoparticles or fluorescent particles) where the signal generated is proportional to the approximate concentration of AMH in a whole blood sample. In particular embodiments, the present invention provides quantitative or semi-quantitative lateral flow immunoassay devices and kits for detecting AMH at home or at the point of care (e.g., in order for women to estimate their ovarian age).

In some embodiments, the present invention provides methods of determining the approximate concentration of Anti-Mullerian hormone (AMH) in a blood sample (e.g., capillary whole blood sample) comprising: a) contacting a whole blood sample from a subject with first antibodies specific for AMH under conditions such that a signal is generated that is proportional to the approximate concentration of AMH in the whole blood sample; and b) detecting the approximate level of the signal, thereby determining the approximate concentration of AMH in the whole blood sample and/or determining the approximate ovarian age of the woman who was the source of the whole blood sample.

In particular embodiments, the first antibodies are labeled with first nanoparticles that produce a colorimetric signal when aggregated (e.g., gold nanoparticles). In certain embodiments, the methods further comprise contacting the whole blood sample with a second antibodies specific for a non-AMH protein in whole blood. In additional embodiments, the second antibodies are labeled with first nanoparticles that produce a colorimetric signal when aggregated (e.g., gold nanoparticles). In other embodiments, the second antibodies are labeled with fluorescent particles that produce a signal when aggregated and visualized with light of a certain wavelength.

In certain embodiments, the contacting is conducted on a membrane, wherein the membrane comprises: at least one test capture region which comprises third antibodies specific for AMH or the first antibodies. In some embodiments, the membrane further comprises: a control capture region which comprises fourth antibodies specific for the non-AMH protein (e.g., any protein present in blood that can serve as a control, such as IgG) or the second antibodies. In other embodiments, detecting the level of the signal comprises detecting the fluorescence absorbance level, the colorimetric intensity level, or the number of colorimetric symbols from, the signal. In additional embodiments, the methods further comprise comparing the approximate amount of the signal to reference signals of known AMH concentration (e.g., where the reference signals are on a reference card) in order to determine the approximate concentration of AMH in the whole blood sample. In further embodiments, the reference signals of known AMH concentration correspond to measurements from samples consisting essentially of washed red blood cells and known AMH concentrations.

In some embodiments, the signal comprises a colorimetric signal. In further embodiments, the approximate concentration that is determined is represented as a non-numerical value. In further embodiments, the non-numerical value comprises the darkness and/or intensity of a colorimetric signal. In certain embodiments, the whole blood sample has a volume of 1 drop of whole blood or less (e.g., ¾ of a drop or ½ of a drop). In particular embodiments, the whole blood sample has a volume of 3-50 ul (e.g., 5 . . . 15 . . . 25 . . . 40 . . . or 50 ul). In certain embodiments, the whole blood sample comprises oxygenated whole blood.

In particular embodiments, the whole blood sample comprises a dried blood sample. In other embodiments, the contacting and detecting are performed with a lateral flow immunoassay device. In particular embodiments, the antibody comprises a Fab fragment or a F(ab′)2 fragment.

In some embodiments, the approximate concentration of AMH detected in the whole blood sample (e.g., using the lateral flow immunoassay devices and kits described herein) is greater than 3.5 ng/ml, wherein the subject is a female, and wherein the method further comprises at least one of the following steps: i) informing said subject that she has, or likely has, polycystic ovarian syndrome; ii) preparing and/or transmitting an electronic and/or paper report that indicates said subject has, or likely has, polycystic ovarian syndrome; iii) preparing and/or transmitting an electronic and/or paper report that said subject should be further evaluated for polycystic ovarian syndrome; iv) prescribing medication and/or surgical treatment to said subject to treat polycystic ovarian syndrome; and v) treating said subject with medication (e.g., metformin or thiazolidinedione (glitazones)), or surgical treatment directed toward alleviating polycystic ovarian syndrome.

In some embodiments, the present invention provides a lateral flow immunoassay device for detecting Anti-Mullerian hormone (AMH) in capillary whole blood comprising: a) a sample pad configured for receiving and transmitting a whole blood sample; b) a conjugate pad in contact with the sample pad and configured for receiving the whole blood sample from the sample pad, wherein the conjugate pad comprises: i) first antibodies specific for AMH, wherein the first antibodies are labeled with first nanoparticles that produce a first colorimetric signal when aggregated, ii) second antibodies specific for a non-AMH protein (e.g., IgG or other abundant protein) in whole blood, wherein the second antibodies are labeled with second nanoparticles that produce a second colorimetric signal when aggregated; c) a membrane in contact with the conjugate pad and configured to receive the whole blood sample from the conjugate pad, wherein the membrane comprises: i) at least one test capture region which comprises third antibodies specific for the AMH or the first antibodies, and, in certain embodiments, ii) a control capture region which comprises fourth antibodies specific for the non-AMH protein or the second antibodies; and d) a substrate (e.g., planar substrate), wherein the sample pad, the conjugate pad, the membrane, and the wick component are supported by the substrate.

In certain embodiments, the device further comprises a wick component in contact with the membrane and configured to absorb excess whole blood sample. In further embodiments, the device further comprises a wicking pad in contact with, and beneath, the sample pad and configured to absorb excess of the whole blood sample such that the sample pad only transmits a set amount (e.g., the same amount of blood is transmitted no matter how much excess blood is added to the sample pad) of the whole blood sample to the conjugate pad. In certain embodiments, the conjugate pad, the membrane, and the wick component are attached to the substrate (e.g., planar substrate). In further embodiments, the first nanoparticles comprise gold nanoparticles or fluorescent particles. In additional embodiments, the second nanoparticles comprise gold nanoparticles or fluorescent particles.

In some embodiments, the at least one test capture region comprises at least two test capture regions (e.g., 2, 3, 4, 5, 6, 7, 8, 9, 10 or more test capture regions). In further embodiments, the third antibodies are present in the at least one test capture region at an excess level compared to the maximum level of AMH that could be present in the amount of whole blood that could reach the at least one test capture region. In further embodiments, the intensity of the first colorimetric signal is proportional to the concentration of AMH present in the whole blood sample.

In additional embodiments, the membrane comprises a nitrocellulose membrane. In other embodiments, the second antibodies are specific for IgG. In certain embodiments, the sample pad comprises a material selected the group consisting of: cellulose, glass, fiber, and polyester. In some embodiments, the sample pad comprises a dried buffer. In particular embodiments, the conjugate pad comprises a material selected from the group consisting of: glass fiber, polyester, and rayon. In other embodiments, the at least one test capture region is in the shape or a line, circle, or oval (or other shape), and wherein the control capture region is in the shape of a line, circle, or oval (or other shape). In further embodiments, the device further comprises a blood reservoir located on top of the sample pad, wherein the blood reservoir is configured to receive a dried blood sample.

In certain embodiments, the components for quantifying AMH in whole blood (e.g., blood reservoir, capture and detection antibodies, conjugate signal, components for moving sample and reagents through the assay system using, for example, cassettes, cards, or chips) and/or a complete lateral flow immunoassay device will be contained within (used in) an electronic device (e.g., lab in a box, or portable detector), to be used for point-of-care testing in clinics, pharmacies, shopping malls, or other locations. In certain embodiments, the electronic device used at point-of-care device uses the principles of lateral flow immunoassay.

In certain embodiments, the present invention provides kits comprising: a) a lateral flow immunoassay device as described herein, and b) at least one component selected from the group consisting of: i) at least one sterile lancet, ii) a gas impermeable foil bag, iii) a color chart, wherein the color chart allows a user of the lateral flow immunoassay to estimate the concentration of AMH in a whole blood sample tested on the lateral flow immunoassay device by comparison to the color chart, iv) a sterile gauze pad, v) a skin sterilization wipe, vi) printed instructions for collecting blood and applying it to the lateral flow immunoassay device, vii) printed instructions for interpreting the first colorimetric signal, viii) a piece of filter paper for collecting a dried blood sample, and ix) a container for housing the lateral flow immunoassay device.

In some embodiments, the at least one component comprises at least two, at least three, at least four, at least five, or at least six of the components. In other embodiments, the at least one component comprises the gas impermeable foil bag, and wherein the lateral flow immunoassay device is located inside the gas impermeable foil bag. In further embodiments, the at least one sterile lancet comprises two sterile lancets.

In particular embodiments, the present invention provides methods of using a lateral flow immunoassay device for detecting Anti-Mullerian hormone in whole blood comprising: applying a whole blood sample to the sample pad of a lateral flow immunoassay device as described herein under conditions such that at least a portion of the whole blood sample migrates from the sample pad, through the conjugate pad to the at least one test capture region and the control capture region in the membrane thereby generating the first and second colorimetric signals, wherein the first colorimetric signal is proportional to the approximate concentration of AMH in the whole blood sample.

In certain embodiments, the methods further comprise detecting the approximate level of the first colorimetric signal, thereby determining the approximate concentration of AMH in the whole blood sample. In further embodiments, the detecting the approximate level of the first colorimetric signal comprises comparing the intensity of the first colorimetric signal to a color chart comprising a plurality of color intensities correlated to different concentrations of AMH. In additional embodiments, the at least one test capture region comprises between 2 and 10 test capture regions, and wherein the method further comprises detecting the number of the capture regions that provide the first colorimetric signal, thereby determining the approximate concentration of AMH in the whole blood sample and/or determining the approximate ovarian age of the woman who was the source of the whole blood sample.

In some embodiments, the lateral flow immunoassay device further comprises a wicking pad in contact with (e.g., and beneath) the sample pad, and wherein the wicking pad absorbs excess of the whole blood sample that is applied to the sample pad such that the sample pad only transmits a set amount of the whole blood sample to the conjugate pad (e.g., the same amount is transmitted to the conjugate pad regardless of whether excess sample is added to the sample pad). In particular embodiments, the lateral flow immunoassay device further comprises a blood reservoir located on top of the sample pad, and wherein the whole blood sample comprises a blood sample (e.g., dried blood sample or liquid blood sample) that is inserted into the blood reservoir, and wherein the method further comprises adding elution or mobilization buffer to the blood reservoir such that blood from the blood sample moves down into the sample pad.

In some embodiments, the present invention provides methods comprising a) detecting the level of AMH in a sample from a subject, where the level of AMH is greater than 3.5 ng/ml, wherein the subject is a female, and b) at least one of the following steps: i) informing said subject that she has, or likely has, polycystic ovarian syndrome; ii) preparing and/or transmitting an electronic and/or paper report that indicates said subject has, or likely has, polycystic ovarian syndrome; iii) preparing and/or transmitting an electronic and/or paper report that said subject should be further evaluated for polycystic ovarian syndrome; iv) prescribing medication and/or surgical treatment to said subject to treat polycystic ovarian syndrome; and v) treating said subject with medication (e.g., metformin or thiazolidinedione (glitazones)), or surgical treatment directed toward alleviating polycystic ovarian syndrome.

### DESCRIPTION OF THE FIGURES

FIG. 1 shows a scatterplot and Passing-Bablok regression analysis of the association between AMH concentrations obtained from matched serum and dried blood spot (DBS) samples for n=78 reproductive age women as described in Example 1.

FIG. 2 shows an association between age and mean AMH concentration in DBS samples from n=78 reproductive age women as described in Example 1.

FIG. 3 shows the stability of AMH when collected and stored in DBS samples. Concentrations of AMH do not increase or decrease substantially for samples stored at room temperature for at least two weeks.

FIG. 4 outlines the general concepts of a lateral flow immunoassay, including a sample pad, conjugate pad, nitrocellulose membrane, wick, test line, and control line.

FIG. 5 shows an exemplary lateral flow immunoassay device of the present invention. FIG. 5A shows sample pad (for receiving blood) located over the top of the wicking pad (for absorbing excess blood). FIG. 5B shows the sample pad moved to the right in order to load the sample with a set volume of blood. In certain embodiments, once the sample pad is moved, elution buffer (e.g., from a dropper bottle) is applied in order to mobilize or further mobilize the blood.

FIG. 6A shows an exemplary embodiment of a lateral flow immunoassay device of the present invention, including a blood reservoir over the top of the sample pad for receiving a blood sample. In certain embodiments, one could apply finger stick blood to a specimen collection card and let it dry. A card punch (also pictured in FIG. 6A) could be then be used to remove a set blood volume. This dried blood sample could then be placed in the blood reservoir and elution/mobilization buffer could be added to cause the sample to liquefy and travel down to the sample pad. Any additional dried blood samples on the specimen collection card could then be mailed in for a laboratory test to confirm the results generated with the lateral flow immunoassay device. FIG. 6B shows an additional exemplary embodiment of a lateral flow immunoassay device of the present invention. In certain embodiments, a capillary pipette is used to remove a measured amount of blood (e.g., from a finger as shown in FIG. 6B). The blood is then applied to the sample pad on the device and elution/mobilization buffer is added if needed.

FIG. 7A shows using a color chart for comparison to a result generated with an exemplary lateral flow immunoassay device in order to at least partially quantify the concentration of AMH in a blood sample. In certain embodiments, the device is configured such that the AMH in the blood sample is the limiting reagent (e.g., the AMH antibody is in excess). FIG. 7B shows the use of multiple test lines in order to at least partially quantify the amount of AMH in a sample (e.g., the more colorimetric lines that appear, the greater the concentration of AMH in a blood sample). FIG. 7C shows the use of multiple test lines in a circular format, with the blood reservoir in the center. Sample flows out to each segment to at least partially quantify the amount of AMH (e.g., the colorimetric line that appears corresponds to the approximate concentration of AMH in a blood sample).

### DEFINITIONS

As used herein, the term “immunoglobulin” or “antibody” refers to proteins that bind a specific antigen. Immunoglobulins include, but are not limited to, polyclonal, monoclonal, chimeric, and humanized antibodies, Fab fragments, F(ab′)2 fragments, and includes immunoglobulins of the following classes: IgG, IgA, IgM, IgD, IgE, and secreted immunoglobulins (sIg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, immunoglobulins, including polyclonal, monoclonal, chimeric, and humanized antibodies; Fab fragments, F(ab′)2 fragments, and Fab expression libraries; and single chain antibodies. An antigen binding protein that is not an antibody can be used in place of an antibody in the methods, compositions, devices and kits of the present invention.

### DETAILED DESCRIPTION

The present invention provides methods, kits, compositions, and devices for detecting Anti-Mullerian hormone (AMH) in biological samples, such as whole blood samples, serum samples, and plasma samples. In certain embodiments, the methods, kits, compositions, and devices employ immunoassays that generate a colorimetric or fluorescent signal (e.g., using antibodies conjugated to gold nanoparticles or fluorescent particles) where the signal generated is proportional to the approximate concentration of AMH in a sample. In particular embodiments, the present invention provides quantitative or semi-quantitative lateral flow immunoassay devices and kits for detecting AMH at home or point of care (e.g., in order for women to estimate their ovarian age). In certain embodiments, the point of care is a doctor's office (e.g., not associated with a laboratory), a drug store (e.g., Walgreen, CVS, RiteAid, etc.), or a retail store (e.g., Walmart, a local mall, etc.)

A. Devices for AMH Detection

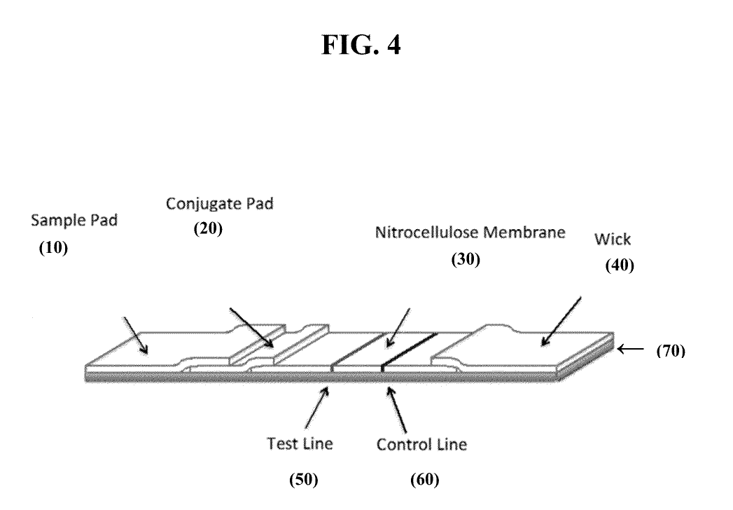

The present invention provides devices, and kits containing devices, for detecting AMH. In certain embodiments, the devices are lateral flow immunoassay devices. An exemplary lateral flow immunoassay device is shown in FIG. 4.

FIG. 4 outlines the general components of a lateral flow immunoassay device. FIG. 4 shows a sample pad (10) for receiving a sample (e.g., whole, oxygenated blood). The sample pad (10) may be composed of any suitable material including, for example, cellulose, glass fiber, polyester, or other filtration materials. In certain embodiments, the sample pad (10) is pretreated with buffer to control pH. The lateral flow immunoassay device further includes a conjugate pad (20) that contains anti-AMH antibody that may be coupled to gold nanoparticles or other chromogenic or fluorescent moiety. The conjugate pad (20) also generally contains control antibodies to a control protein (e.g., IgG) normally present in whole blood. Such control antibodies may also be conjugated to gold or other chromogen or fluorescent moiety. The conjugate pad (20) may be composed of any suitable material including, for example, glass fiber, polyester, or rayon. The conjugate pad (20) may be pretreated with proteins, surfactants, and polymers to ensure consistent nanoparticle release. The lateral flow device also comprises a membrane (30) (e.g., nitrocellulose membrane) that contains a test line/symbol and a control line/symbol. The membrane (30) is chosen for capillary flow and high protein binding capacity. Preferably, the membrane (30) is low cost and is available in various wicking rates. The test line or symbol (50) in the membrane generally contains secondary antibodies specific for AMH or the anti-AMH antibodies in the conjugate pad. The control line or symbol (60) in the membrane contains capture antibodies specific for the control antibodies and is used to ensure the device is working properly. In certain embodiments, the text and control lines (or symbols, like a circle or plus or minus sign) are blocked to control flow rates and to stabilize proteins. The lateral flow device may also contain, in certain embodiments, a wick (40) at the end which absorbs excess sample and/or buffer and prevents back flow. The various components of the lateral flow immunoassay device are supported by a substrate (70) (e.g., a planar substrate).

In certain embodiments, the lateral flow immunoassay devices are used as follows. A drop of blood is collected using a finger stick from a sterile, single use lancet. Blood is collected onto the sample pad (10), where it will be drawn into the conjugate pad (20) by capillary action. In the conjugation pad (20), the AMH in the blood sample reacts with anti-AMH antibodies (e.g., conjugated to gold nanoparticles or fluorescent particles) and control protein antibodies (e.g., that are conjugated to gold nanoparticles or fluorescent particles). The bound analytes then travel through the sample membrane (30) until captured by a secondary antibody (e.g., anti-AMH, or antibodies to the anti-AMH antibodies, or antibodies to the control antibody) imbedded into the test line(s) (50) and control line (60). The intensity of the color (or fluorescent signal) produced by the gold nanoparticles (or fluorescent particles) is proportional to the bound AMH concentrations, and thus can be used to directly estimate AMH blood concentrations.

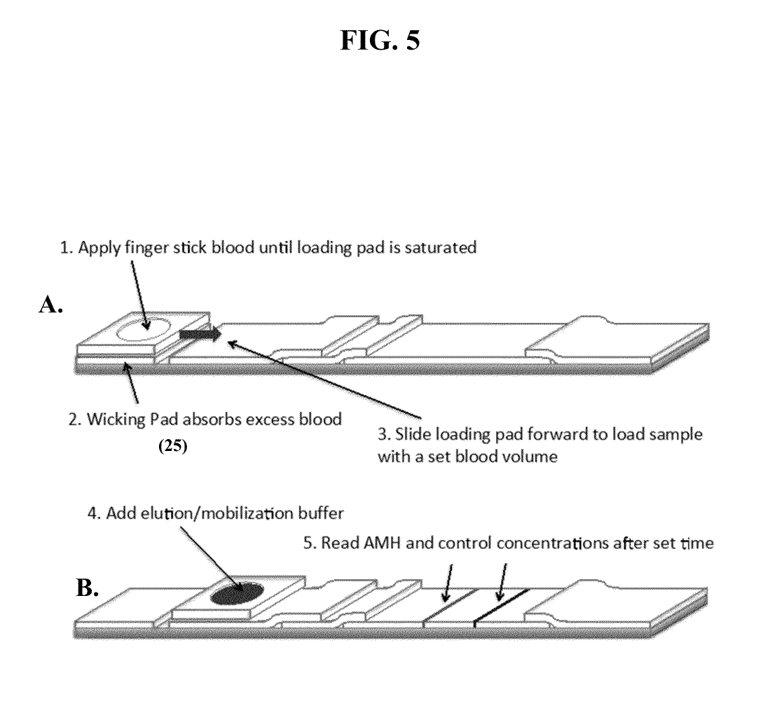

FIG. 5 outlines one embodiment of a lateral flow immunoassay device for controlling the volume of blood that is applied to the device. The application of a controlled volume of blood is generally important for a semi-quantitative assay, since the concentration of AMH is dependent on the volume of blood applied to the test. FIG. 5A shows an embodiment where the sample pad (20) is over a wicking pad (25) when the blood is applied. In this configuration, excess blood can be collected by the wicking pad. FIG. 5B shows the sample pad slid over to the right to start the blood (or blood with buffer added) flowing through the assay.

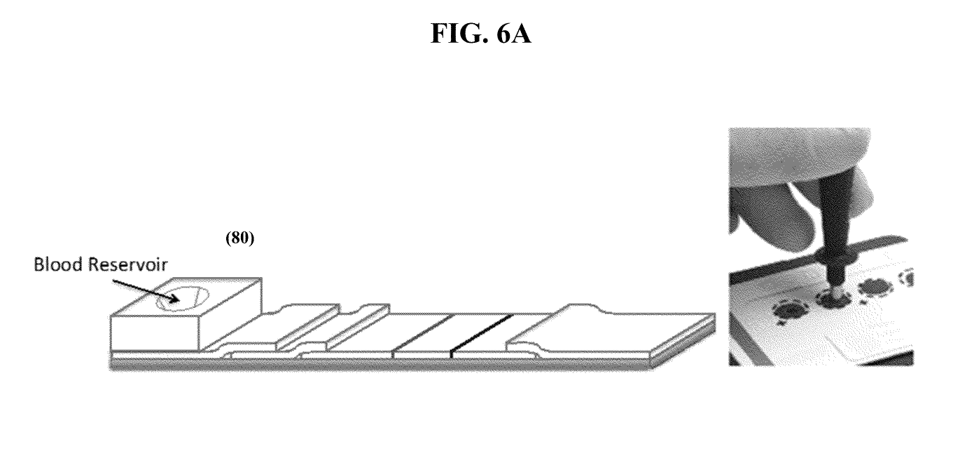

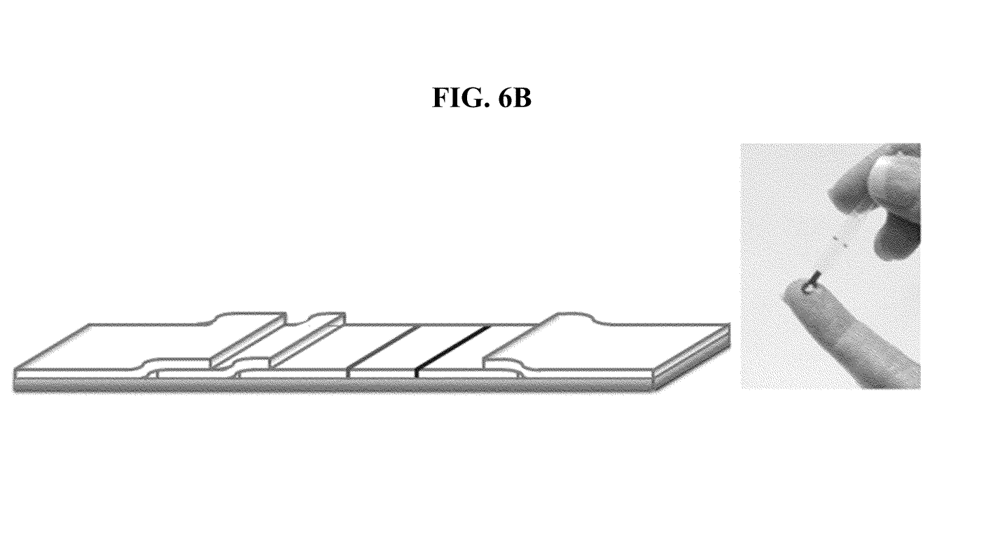

FIG. 6 outlines the principles of embodiment of a lateral flow device for controlling blood volume. FIG. 6A shows an exemplary embodiment of a lateral flow immunoassay device of the present invention, including a blood reservoir (80) over the top of the sample pad for receiving a dried blood sample. In certain embodiments, one could apply finger stick blood to a specimen collection card and let it dry. A card punch (also pictured in FIG. 6A) could be then be used to remove a set blood volume. This dried blood sample could then be placed in the blood reservoir and elution/mobilization buffer could be added to cause the sample to liquefy and travel down to the sample pad. Any additional dried blood samples on the specimen collection card could then be mailed in for a laboratory test to confirm the results generated with the lateral flow immunoassay device. FIG. 6B shows an additional exemplary embodiment of a lateral flow immunoassay device of the present invention. In certain embodiments, a capillary pipette is used to remove a measured amount of blood (e.g., from a finger as shown in FIG. 6B). The blood is then applied to the sample pad on the device and elution/mobilization buffer is added if needed.

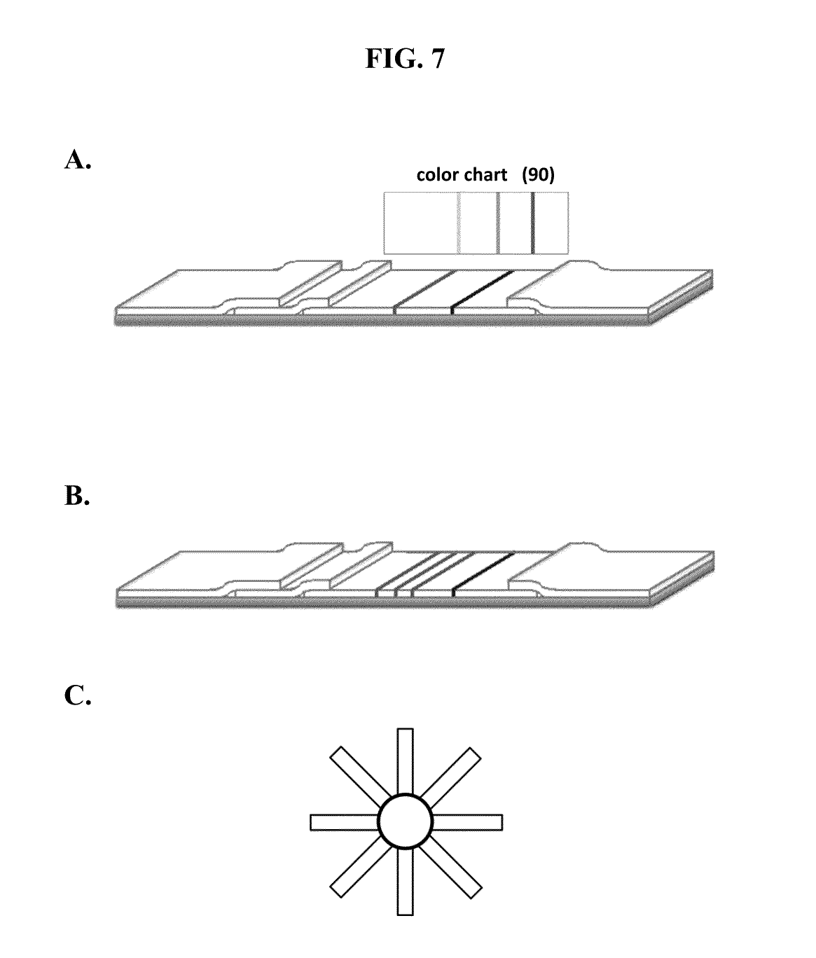

FIG. 7 shows two exemplary embodiments for AMH detection. The first design, in FIG. 7A, is based on measuring a color intensity on the test line (50), which is subsequently compared to a color chart (90) to estimate AMH concentrations. The second design (FIG. 7B) includes multiple test lines, so that AMH concentrations are estimated by the number of lines where color develops, rather than absolute intensity. The third design (FIG. 7C) uses a circular format, where sample flows from the blood reservoir to separate membranes, each of which has different concentration of capture antibodies which are calibrated to develop color at different concentrations of AMH. The colorimetric line that appears corresponds to the approximate concentration of AMH in the sample.

In certain embodiments, the lateral flow immunoassay devices and configured as kits with additional components, and, in particular embodiments, are configured for in-home use (e.g., configured for sale at a drug store or other outlet) or configured for use at a point of care. In some embodiments, the devices and related composed are contained within a labeled box (e.g., approximately 6 (h)×4 (w)×1.5 (d) inches; attractively labeled). In particular embodiments, the box contains materials for blood collection (e.g., two sterile lancets, an alcohol prep, a sterile gauze pad). In certain embodiments, the box contains instructions (e.g., easy to read) for collecting blood and applying it to the device. In further embodiments, the box contains the device which is sealed in a gas-impermeable foil bag. In some embodiments, the box contains easy-to-read instructions for interpreting the results of the test (e.g., instructions for correlating AMH concentration with ovarian age). In further embodiments, the box contains a separate piece of filter paper, upon which another drop of blood is applied at the same time blood is applied to the device. This sample would then be placed into a different gas impermeable foil bag with desiccant, and mailed to a lab for more precise measurement of AMH (e.g., postage paid envelope included). In particular embodiments, women employing the device can refer to an internet page for more information on how to interpret their test results and what options they can pursue if they are concerned about the results.

In certain embodiments, in parallel with the home-based AMH test, an additional lancet and specimen collection card could be included with the home test kit to provide the option to mail in a DBS (dried blood) sample for more accurate quantitation. For example, while the home test may only provide a semi-quantitative estimation of AMH levels (e.g., low, medium, high), the mail-in test could provide an accurate determination of AMH, comparable to the current clinical “gold standard” serum method.

In certain embodiments, the chemistry in the lateral flow immunoassay device is a competitive assay as follows. The conjugate pad may contain AMH-nanoparticle conjugate (note that the conjugate, such as gold nanoparticles, is bound to the AMH in this assay, not the antibody). The pad would also contain anti-AMH antibody (not conjugated to anything). When sample is added, the AMH in the sample will bind to AMH antibody. The AMH-conjugate will also bind to AMH antibody. Both complexes will flow to the capture zone. Anti-AMH antibody will capture the complexes (note that the anti-AMH antibody in the capture zone will bind to constant regions of the AMH antibody). Color change will be negatively proportional to the concentration of AMH in the sample (i.e., more AMH, less color development). This is due to competition between the AMH-antibody and AMH-conjugate-antibody complexes for binding sites in the capture zone.

B. Dried Blood Spot Detection of AMH

In certain embodiments, AMH is detected from a dried blood spot, using, for example, the devices herein or assays similar to that described in Example 1 below.

Dried blood spots (DBS)—drops of whole blood collected on filter paper following a simple finger stick—represent a minimally-invasive alternative to venipuncture blood collection (see, e.g., McDade, et al., 2007, herein incorporated by reference in its entirety). Generally, the participant's finger is cleaned, pricked with a sterile, disposable lancet of the type commonly used to monitor blood glucose, and drops of whole blood are applied to the paper. Samples are allowed to dry, and then generally stacked and stored in plastic bags prior to shipment to the laboratory. A major advantage of DBS sampling is that is relatively painless and non-invasive, low cost, and can be implemented by non-medically trained personnel in the participant's home or other non-clinical setting (e.g., drug store, large retail store, a mall, etc.). The simplified logistics of DBS sampling allow investigators to collect blood from large numbers of participants in diverse research settings, and over the past five years more than 35,000 DBS samples have been collected as part of major health surveys in the US (McDade, et al. 2007).

Example 1 below provides methods for quantifying AMH in DBS samples (e.g., in order to promote research on the causes and consequences of variation in ovarian reserve in a wider range of research settings). Following previously validated and widely used DBS assay protocols (McDade, et al., 2004, McDade and Shell-Duncan, 2002, McDade, et al., 2000), Example 1 provides methods for detecting AMH from DBS. Example 1 reports on results of assay validation demonstrating levels of performance in the quantification of AMH that are comparable to gold-standard, serum-based methods. As described in Example 1, matched serum and DBS samples were obtained from n=78 reproductive- age women. There was strong agreement between AMH concentrations measured in DBS and serum samples across the entire assay range. Analysis of within-assay (percent coefficient of variation, 4.7-6.5%) and between-assay (3.5-7.2%) variability indicated a high level of assay precision and reliability, respectively.

The minimum detectable dose of AMH was 0.052 ng/mL. Concentrations of AMH remained stable in DBS samples stored for at least two weeks at room temperature, and for four weeks when refrigerated. These results indicate that the DBS assay performs at a level that is comparable to serum-based methods, with the advantage of lower burdens and costs associated with blood collection that may be advantageous for epidemiologic research on the causes and consequences of variation in ovarian reserve.

C. AMH, Ovarian Reserve, and Home Based Testing

In certain embodiments, the AMH levels detected by the devices, systems, kits, and methods of the present invention are used to determine a women's ovarian reserve. Ovarian reserve is important because it is related to a woman's ability to conceive, and to the timing of menopause. In certain embodiments, the present invention allows a woman to determine the “age” of her ovaries, which would allow her to plan her reproductive future. This is important as recent socio-demographic and economic trends show that women in the U.S., and most other developed nations, are waiting longer to have children, and are therefore running up against natural limitations in their ability to conceive. This is evidenced by the fact that 12% of U.S. women now seek consultation for infertility.

The relevance of such a home test is highlighted by several socio-demographic and economic trends. Women in the U.S. are waiting longer to have children so they can pursue education and career opportunities, while higher rates of divorce have also encouraged women to begin families later in life. The number of first births to women over 30 has increased fourfold (5% to 24%) since 1975 (Macaluso, et al., 2008). This trend is running up against natural age-related limitations in a woman's ability to conceive. As a result, there has been a parallel increase in the number of women seeking medical consultation for infertility, which affects 7.3 million people in the US (12% of women of childbearing age, or 1 in 8 couples). Every woman is born with a fixed number of eggs in her ovaries, and this number decreases over time. The number of eggs at any one point in time reflects a woman's “ovarian reserve.”

Anti-Müllerian hormone—also called Müllerian inhibiting substance—has recently emerged as a clinically important biomarker of ovarian reserve. AMH is a glycoprotein dimer produced by granulosa cells from small pre-antral and antral follicles in the ovary, and prior studies indicate that AMH represents a useful measure of ovarian reserve since it plays an important role in early stage follicle development, can be quantified in serum or plasma, and because levels fluctuate minimally during the menstrual cycle (Fauser, et al., 2002, Somunkiran, et al., 2007). Concentrations show a progressive decrease with age (Lee, et al., 1996, Seifer, et al., 2011) and predict timing of menopause in advance of clinical symptoms (e.g., menstrual irregularity) (Tehrani, et al., 2009, van Rooij, et al., 2004). In addition, reduced AMH has been associated with lower likelihood of conception (Steiner, et al., 2011), and among women undergoing ovarian stimulation for in vitro fertilization, AMH predicts ovarian response and pregnancy (Seifer et al, 2002, van Rooij, Tonkelaar, Broekmans, Looman, Scheffer, de Jong, Themmen and to Velde, 2004, McIlveen, et al., 2007, Kwee, et al., 2008, van Rooij, et al., 2002).

Presently, consultation with a reproductive endocrinologist is required to accurately assess a woman's ovarian reserve (egg quantity and quality), which is a critical variable in determining a woman's likelihood of conception. Clinically, the physician orders venipuncture blood samples for a battery of tests (including AMH), and an ultrasound, and uses this information to make a judgment regarding a woman's ovarian reserve. Typically this assessment takes two or more office visits (2 to 3 visits) to accomplish. Of all of the tests (e.g., serum FSH, E2, inhibin B, AMH) typically performed, AMH is the earliest, most sensitive, least variable and most convenient to obtain as it can be done anytime during the menstrual cycle, does not require additional expertise and is not affected by the presence of other commonly used medications such as oral contraceptives. In addition, there is every indication that AMH is useful for predicting the timing of the onset of menopause.

In certain embodiments, the clinical significance of AMH concentration results (e.g., from a DBS and/or lateral flow immunoassay) are determined by referring to an algorithm (e.g., posted on a website) that compares the users AMH result with population based age-specific ranges (see, e.g., Seifer et al, 2011, herein incorporated by reference) . This will allow the user to know, for example, if their AMH level is within 1 or 2 or 3 standard deviations of the mean/median for their specific chronologic age or if they are at the mean/median for a younger or older age. The algorithm can provide probabilities of number of years before one could expect the onset of menopause based on current age and AMH level (see, e.g., Broer et al, 2011, herein incorporated by reference) derived from DBS testing. In certain embodiments, detecting AMH levels will allow a woman to determine her approximate age of menopause, for example, using Table 2 of Tehrani et al. (J. Clin. Endocrin Metab., Feb. 2013, 98(2), pages 1-6), which is herein incorporated by reference in its entirety, including specifically Table 2. For example, in Table 2 of Tehrani et al., if a 24 year old woman is found to have an AMH level of 1.7 ng/dL (e.g., using the devices and methods herein) her average age of menopause can be estimated at 45 years old. A recommendation regarding the advisability of seeking medical assistance in addition to specific questions to ask a user's physician could be provided to assist the user in receiving informative information regarding choices of life style and reproductive options.

The availability of an at-home test for AMH would provide women with important information for family planning, and for preparing for a postmenopausal stage of life. Currently this information can only be obtained as part of an intensive clinical work-up, with a referral to a reproductive endocrinologist. The usefulness of an at-home test for AMH would also have worldwide appeal as governments in countries such as Germany, France, Russia, Italy, Japan continue to be concerned over their aging populations and reductions in fertility. Many of these countries have spoken of taking steps in the future to promote family building in an effort to replete their aging populations. Access to an at-home test could assist in these efforts while lowering the medical cost of fertility care across the globe for industrialized nations.

### EXAMPLES

### Example 1

Detecting Anti-Mullerian Hormone in Dried Blood Spots Anti-Müllerian hormone (AMH) has emerged as a clinically useful measure of ovarian reserve, but the requirement for venous blood, and serum or plasma, is an obstacle to application in non-clinical settings. This Example describes a new method for quantifying AMH in dried blood spot (DBS) samples, drops of whole blood collected on filter paper following a simple finger stick. Briefly, matched serum and DBS samples were obtained from n=78 women of reproductive age, and AMH values were compared using regression analyses and scatter plots. The precision, reliability, linearity, recovery, and lower detection limit of the DBS assay was evaluated, as well as the stability of AMH in DBS across a range of storage conditions. There was strong agreement between AMH concentrations measured in DBS and serum samples across the entire assay range. Analysis of within-assay (percent coefficient of variation, 4.7-6.5%) and between-assay (3.5-7.2%) variability indicated a high level of assay precision and reliability, respectively. The minimum detectable dose of AMH was 0.052 ng/mL. Concentrations of AMH remained stable in DBS samples stored for two weeks at room temperature, and for four weeks when refrigerated. The DBS assay performs at a level that is comparable to serum-based methods, with the advantage of lower burdens and costs associated with blood collection. This assay may be employed in clinical as well as non-clinical settings on the causes and consequences of variation in ovarian reserve.

### Methods

### Sample Collection

For the purposes of assay validation, a matched set of finger stick DBS samples and venipuncture serum samples were collected from 78 volunteers. Inclusion criteria were as follows: age between 18-45 years, the presence of both ovaries, and a history of regular menstrual cycles 21-35 days in length. Exclusion criteria included pregnancy or lactation within the prior 3 months. The protocol was approved by the Institutional Review Board, and all study participants provided informed consent before inclusion in the study.

Serum and DBS samples were collected from each participant during the same clinic visit. First, approximately 4 mL blood were drawn into a serum separator tube (SST) using standard venipuncture procedures. Each tube was allowed to clot at room temperature in an upright position for 30 minutes and centrifuged at 1000×g for 15 minutes. Serum was then aliquoted into cryovials and frozen at −80° C.

Immediately following venipuncture, finger stick capillary blood samples were collected on filter paper by delivering a controlled, uniform puncture with a sterile, disposable micro-lancet (BD Microtainer #366594, Franklin Lakes, N.J.). After wiping away the initial drop of blood with sterile gauze, up to five drops of whole blood were applied to the filter paper (Whatman #903, GE Healthcare, Piscataway, N.J.), allowed to dry at room temperature for at least 4 hours, and then placed in a gas impermeable plastic bag with desiccant and stored frozen at −30° C.

### Serum AMH Assay Protocol

Concentrations of AMH were determined in serum samples using a recently validated, commercially available enzyme immunoassay kit designed for use with serum or plasma samples (Beckman Coulter #A73818, Brea, Calif.) (Kumar, et al., 2010). Samples were analyzed in duplicate using materials and procedures provided with the kit. Briefly, samples, calibrators, and controls were pipetted into microtiter plate wells coated with anti-AMH capture antibody, incubated for 60 minutes, and then washed five times with a microplate washer (BioTek Instruments ELx50, Winooski, Vt.). Biotinylated anti-AMH detection antibody was then added, wells were incubated for 60 minutes and washed, followed by the addition of streptavidin-horseradish peroxidase (HRP). Wells were incubated for 30 minutes, washed, and tetramethylbenzidine chromogen solution was added to promote color change in proportion to the amount of bound AMH. Sulfuric acid was added to each well to stop the reaction, and absorbance was measured at 450 nm with 630 nm reference wavelength in a microplate reader (BioTek Instruments E1×808, Winooski, Vt.). Sample concentrations were calculated from the best fit 4-parameter logistic standard curve based on absorbance values derived from calibration materials with known concentrations of AMH.

### Dried Blood Spot AMH Assay Protocol

The protocol for analyzing AMH in DBS samples discussed in detail here is based on modifications to the Beckman Coulter AMH enzyme immunoassay kit (Beckman Coulter #A73818, Brea, Calif.). Similar results are possible with other commercially available antibodies and immunoassay kits for quantifying AMH (e.g., Ansh Labs, Webster, Tx., AnshLite™ AMH CLIA, AL-205). In order to minimize matrix differences and maximize comparability between calibration material and samples, DBS standards were manufactured by diluting AMH stock of known concentration with washed erythrocytes, followed by application onto filter paper. Washed erythrocytes were obtained as follows: 1) Whole blood was collected by venipuncture in 5 mL EDTA vacutainer tubes, and centrifuged at 1,500×g for 15 minutes; 2) Plasma and buffy coat were removed and discarded; 3) Approximately 3 mL normal saline (0.86 g NaCl/100 mL deionized H₂O) were added; 4) Tubes were mixed gently for 5 minutes on a hematology rotor and centrifuged as before. Saline and any remaining buffy coat were removed, and steps 3 and 4 were repeated for a total of 3 washes.

DBS AMH standards were made as follows: 1) Stock AMH (Beckman Coulter #A73819, Brea, Calif.) was obtained at concentrations across the likely physiological range (22.5, 10.0, 4.0, 1.2, 0.4, 0.16, and 0.0 ng/mL); 2) Each concentration of AMH stock was added to an equal volume of washed erythrocytes (1:2 dilution); 3) Solutions were mixed gently for 5 minutes on a hematology rotor; 4) Standards were then applied to labeled filter paper cards in 50 μL drops using a manual pipette, dried overnight at room temperature, and stored at −30° C. in gas impermeable plastic bags with desiccant. Final DBS AMH standard concentrations were 11.25, 5.0, 2.0, 0.6, 0.2, 0.08, and 0.0 ng/mL. DBS-based control samples with low and high AMH levels were also manufactured using these procedures.

The day before an assay was to be performed, DBS standards, samples, and controls were removed from the freezer, and discs were punched out using a 3.2 mm (⅛ inch) hole punch and placed into a 96-well filter plate for overnight elution (MultiScreen HTS, Millipore #MSHVN4510, Billerica, Mass.). For duplicate measures, three discs were placed in two separate wells, for a total of six discs. Assay buffer (100 uL) was added to each well, the plate was covered, and then incubated overnight at 4° C. The day of the assay, the plate was removed from refrigeration and placed on an orbital plateshaker at 250 rpm for 30 minutes. The filter plate was then stacked on top of the assay plate (pre-coated with anti-AMH capture antibody) provided with the kit, and centrifuged for two minutes at 2,100×g. This elution protocol maximizes the recovery of sample since all material flows through the filter plate directly into the assay plate; no material is wasted due to pipetting, and filter paper discs are efficiently removed from the sample.

The DBS assay was then performed as follows: 1) The assay plate was incubated with shaking (700 rpm) for 90 minutes and then washed five times with a microplate washer (BioTek Instruments EL×50, Winooski, Vt.); 2) Biotinylated anti-AMH detection antibody (100 uL) was added to each well, the plate was incubated with shaking for 90 minutes, and washed five times; 3) Streptavidin-HRP (100 uL) was added to each well, the plate was incubated with shaking for 30 minutes, and washed five times; 4) Chromogen solution (100 uL) was added to each well and the plate was incubated with shaking for 15 minutes away from direct light exposure; 5) Sulfuric acid stop solution (100 uL) was added to each well, and absorbance was measured at 450 nm (630 nm reference) in a microplate reader (BioTek Instruments E1×808, Winooski, Vt.). Sample concentrations were calculated from the best fit 4-parameter logistic standard curve based on absorbance values derived from DBS standards with known concentrations of AMH.

### Analysis of Assay Performance

The performance of the DBS assay was investigated by evaluating agreement between DBS and serum AMH concentrations in matched samples, as well as linearity, recovery, precision and reliability, and lower detection limit. In addition, the stability of DBS AMH was investigated under a range of storage conditions. Statistical analyses were performed using Excel (2007, Microsoft Corp., Redmond, Wash.) and STATA (version 11.1; STATA Corp, College Station, TX). The strength and linear dependence of results derived from matched DBS and serum samples was investigated with Pearson correlation and Passing-Bablok regression, as well as inspection of Bland-Altman plots for evidence of bias or inconsistent variability across the range of measurement (Bland and Altman, 1999, Bland and Altman, 1986).

Linearity of dilution was assessed by eluting and then serially diluting (1:2, 1:4, 1:8, 1:16) two DBS samples at the high end of the assay range. Recovery was evaluated with two control sera containing low (2.5 ng/mL) and high (8.0 ng/mL) concentrations of AMH which were diluted 1:2 with washed erythrocytes and spotted onto filter paper. For the analysis of linearity and recovery, observed values were compared to expected values and multiplied by 100 for a measure of % recovery. Assay precision and reliability were evaluated by calculating within-assay and between-assay coefficients of variation (CV; standard deviation/mean) from multiple determinations of two laboratory controls at the low and high end of the assay range, respectively. Precision was evaluated with 10 determinations of each control in a single assay, and reliability was evaluated with duplicate measurements of each control across ten assays performed on different days.

Lower detection limit (minimum detectable dose) was evaluated based on ten determinations of the zero standard (assay buffer and washed erythrocytes) measured on a single assay plate. The mean absorbance of the zero standard was calculated, and the point 2 SD above zero was plotted on the assay standard curve to determine the lowest DBS AMH concentration that could be differentiated from zero with confidence.

The stability of AMH in blood spots was determined over a four week period in which DBS samples from three individuals were exposed to one of three steady temperature conditions (4° C., room temperature (21° C. to 23° C.), 37° C.), and one oscillating condition (12 hours at 32° C. and 12 hours at 21° C. to represent ambient conditions in tropical environments). Samples were considered to be stable so long as values remained within a 2 SD range of initial baseline values (based on the mean and SD of 10 determinations of samples placed in the freezer after drying overnight). Samples were exposed for 1, 2, 3, 4, 5, 6, 7, 14, 21, or 28 days in gas impermeable bags with desiccant, stored at −30° C. after the period of exposure, and then analyzed together along with baseline samples. In addition, the stability of AMH in DBS to repeated cycles of freezing and thawing was also evaluated to consider the potential effects of removing samples from the freezer during assay set up. Three DBS samples were removed from their plastic bags and placed on the benchtop at room temperature for 1 hour, and then returned to the freezer. The procedure was repeated over five different days.

### RESULTS

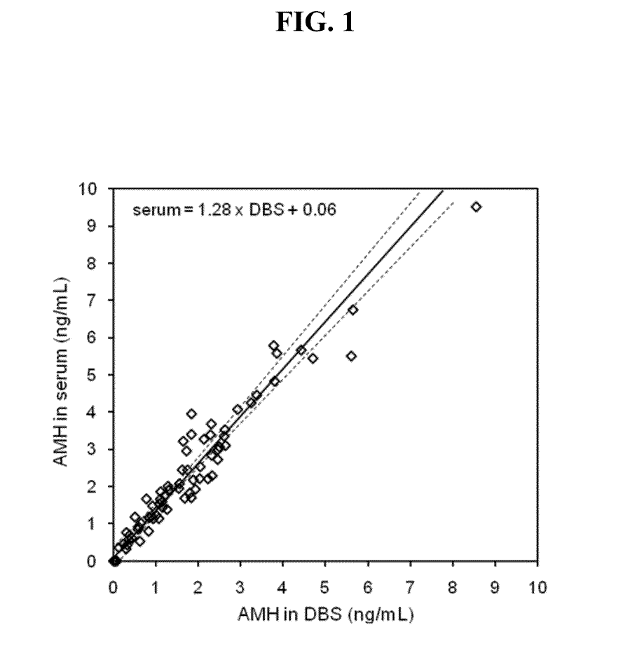

Mean age of women providing validation samples was 29.9 (SD 8.1) years, and mean BMI was 26.0 (SD 6.8). Analysis of paired DBS and serum samples revealed a high level of agreement in AMH results across the two assays (FIG. 1). The association between DBS and serum values was strong and linear across the entire assay range, with Pearson R=0.968. Values from DBS were systematically lower than serum values, consistent with previously developed DBS methods (McDade, Burhop and Dohnal, 2004, McDade, Stallings, Angold, Costello, Burleson, Cacioppo, Glaser and Worthman, 2000): mean DBS AMH concentration was 1.70 ng/ml (SD 1.50), and mean serum concentration was 2.23 ng/mL (SD 1.80). This result is expected since serum and DBS samples are comprised of distinct matrices, and the diluting effect of erythrocytes typically results in lower analyte concentrations in DBS samples. The regression equation in FIG. 1 provides a means for estimating serum equivalent values from DBS results if desired (Worthman and Stallings, 1994).

Serum and DBS results were also compared by calculating the ratio of serum AMH to DBS AMH, and visually inspecting for evidence of bias or inconsistent variability across the range of measurement (Bland and Altman, 1999, Bland and Altman, 1986). The mean ratio was 1.36 (SD=0.47), with three values lying outside the 95% limits of agreement. These values belonged to samples with DBS AMH concentrations <0.5 ng/mL, and reflect slightly higher variability in the agreement between serum and DBS results for samples at the low end of the assay range. There was no evidence of systematic differences in the ratio of serum to DBS results across the assay range.

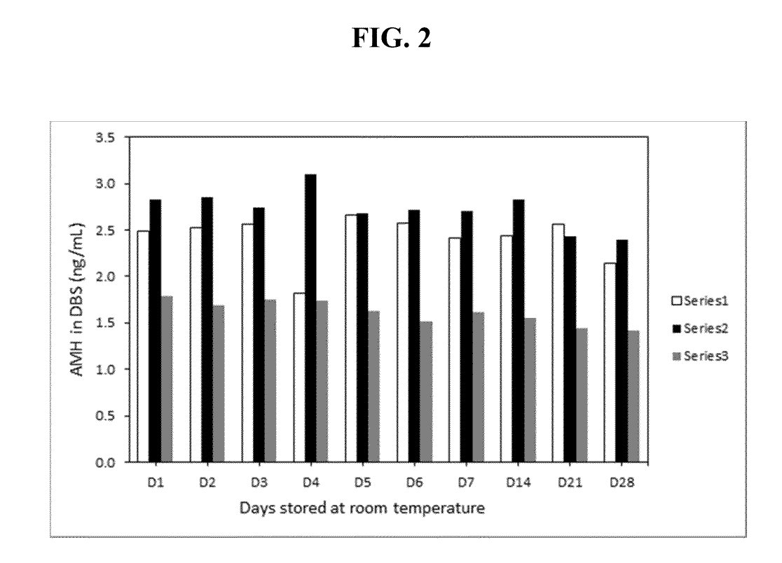

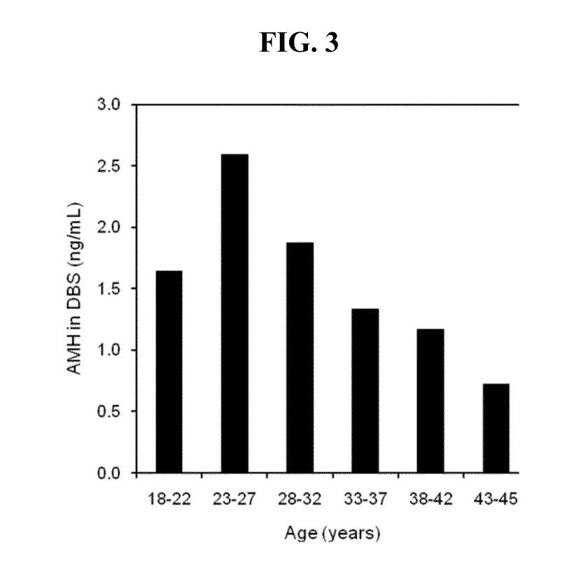

Prior studies of reproductive age women have consistently demonstrated a negative association between age and AMH (Lee, Donahoe, Hasegawa, Silverman, Crist, Best, Hasegawa, Noto, Schoenfeld and MacLaughlin, 1996, Seifer, Baker and Leader, 2011). This Example found a similar decline in DBS AMH concentrations with age in the validation sample, particularly among women 28 years and older (FIG. 2).

Analysis of two serially diluted samples indicated a high degree of assay linearity, with observed values ranging from 83.3% to 102.6% of expected for the first sample (7.72 ng/mL starting concentration), with a mean of 94.1%. Observed values ranged from 92.7% to 98.6% of expected for the second sample (5.55 ng/mL starting concentration), with a mean of 95.4%. Analysis of recovery produced similar results. For low and high control samples, the observed values were 107.7% and 97.1% of expected, respectively.

Repeat analysis of two control samples within and across assay plates indicated a high degree of precision and reliability. Within-assay % CV for the low control (x=1.19 ng/mL) was 6.5%, and between-assay % CV as 7.2%. For the high control (x=3.72 ng/mL), within-assay % CV was 4.7%, and between-assay % CV was 3.5%.

The lower detection limit of the assay was determined to be acceptably low. Based on a criterion of 2 SD above the zero standard, the minimum detectable dose of AMH was 0.052 ng/mL in this Example. A more conservative 3 SD criterion resulted in a minimum detectable dose of 0.075 ng/mL. It is worth noting that in the analysis of paired DBS/serum samples, the DBS assay was able to detect AMH for all women who also had detectable levels of AMH as determined with the serum assay. The lower detection limit of the DBS assay therefore approximates that of the serum assay.

Analysis of AMH stability in DBS indicates that samples can be stored refrigerated at 4° C. for at least four weeks, and for two weeks at room temperature, without significant decreases in AMH concentration compared to baseline. Concentrations of AMH declined after three weeks of storage at room temperature, with values across the samples averaging 89.8% of baseline. Samples remained stable for seven days when exposed to the oscillating condition, and declined to 91.4% of baseline by 14 days. Concentrations of AMH declined rapidly in samples stored at 37° C. By day 3, concentrations were reduced on average to 85.7% of baseline. There was no consistent pattern of degradation in AMH concentrations across five cycles of freezing and thawing.

### DISCUSSION

This Example provides a minimally-invasive method for quantifying AMH in DBS samples (e.g., which can be sued in order to facilitate research on ovarian reserve). Analysis of assay performance indicates that the DBS method returns AMH results that are accurate, precise, reliable, and in strong agreement with the current gold-standard serum-based assay method. In addition, since the protocol uses commercially available supplies and commonly available enzyme immunoassay equipment, barriers to implementation are relatively low.

Previously validated methods for quantifying gonadotropins and steroid hormones in DBS have promoted comparative, community-based research on human reproductive function for almost two decades (Worthman and Stallings, 1994, Worthman and Stallings, 1997). The AMH methods of this Example adds an important biomarker to this methodological toolkit, and takes advantage of the low costs and simplified logistics associated with collecting DBS samples. The finger stick procedure is relatively painless and non-invasive, and has yielded high rates of participant compliance in multiple community- and population-based studies (Borders, et al., 2007, Williams and McDade, 2009, McDade, 2011). For example, in a recent application in a large, nationally representative study of young adults, 94% of participants consented to provide a DBS sample (Harris, 2010).

Requirements for storage and transportation are simplified by the fact that DBS samples can be stacked and stored in air-tight containers and kept at ambient temperatures. Results of the stability analysis indicate that samples can be stored for two weeks at normal room temperature without loss of AMH, and that this period can be extended to four weeks or longer with refrigeration. However, the AMH in DBS is sensitive to elevated temperatures, with more rapid degradation with exposure to temperatures common in tropical regions, and during summer months in other areas. Efforts should therefore be made to protect samples from prolonged exposure to high temperatures during storage and shipping.

Factors to consider for DBS sampling include the following. First, proper placement of whole blood on the filter paper is important, since the dispersion of analytes within the sample will be inconsistent if blood is blotted or smeared onto the paper, or if a drop of blood is placed on top of a previously collected drop. In certain embodiments, the AMH assay may use a relatively large quantity of whole blood: six 3.2 mm discs are required for duplicate analyses, which is the volume that can be obtained from one large drop of blood (˜50 uL). The filter papers used included pre-printed circles as guides for blood placement, and by collecting at least one large drop of blood that fills the border of this circle one can be assured of having enough sample. While the process of collecting DBS samples is relatively straightforward, implementing procedures that ensure sufficient sample volume and that avoid blotting and smearing are important for successful quantification of AMH.

### REFERENCES

Durlinger, A. L. L., Gruijters, M. J. G., Kramer, P., Karels, B., Ingraham, H. A., Nachtigal, M. W., Uilenbroek, J. T. J., Grootegoed, J. A. and Themmen, A. P. N. (2002) Anti-Mullerian hormone inhibits initiation of primordial follicle growth in the mouse ovary. Endocrinology, 143, 1076-1084.

Fauser, B. C. J. M., de Vet, A., Laven, J. S. E., de Jong, F. H. and Themmen, A. P. N. (2002) Antimullerian hormone serum levels: a putative marker for ovarian aging. Fertility and Sterility, 77, 357-362.

Somunkiran, A., Yavuz, T., Yucel, O. and Ozdemir, I. (2007) Anti-Mullerian hormone levels during hormonal contraception in women with polycystic ovary syndrome. Eur J Obstet Gyn R B, 134, 196-201.

Lee, M. M., Donahoe, P. K., Hasegawa, T., Silverman, B., Crist, G. B., Best, S., Hasegawa, Y., Noto, R. A., Schoenfeld, D. and MacLaughlin, D. T. (1996) Mullerian inhibiting substance in humans: Normal levels from infancy to adulthood. J Clin Endocr Metab, 81, 571-576.

Seifer, D. B., Baker, V. L. and Leader, B. (2011) Age-specific serum anti-Mullerian hormone values for 17,120 women presenting to fertility centers within the United States. Fertility and Sterility, 95, 747-750.

Seifer D B, MacLaughlin D T, Christian B P, Feng B, Shelden R M: Early follicular serum mullerian-inhibiting substance levels are associated with ovarian response during assisted reproductive technology cycles. Fertil Steril 77:468-71, 2002.

S. L. Broer, M. J. C. Eijkemans, G. J. Scheffer, I. A. J. van Rooij, A. de Vet, A. P. N. Themmen, J. S. E. Laven, F. H. de Jong, E. R. te Velde, B. C. Fauser, and F. J. M. Broekmans Anti-Mullerian Hormone Predicts Menopause:A Long-Term Follow-Up Study in Normoovulatory Women J Clin EndocrinolMetab 96: 2532-2539, 2011.

Tehrani, F. R., Solaymani-Dodaran, M. and Azizi, F. (2009) A single test of antimullerian hormone in late reproductive-aged women is a good predictor of menopause. Menopause, 16, 797-802.

van Rooij, I. A., Tonkelaar, I., Broekmans, F. J., Looman, C. W., Scheffer, G. J., de Jong, F. H., Themmen, A. P. and te Velde, E. R. (2004) Anti-mullerian hormone is a promising predictor for the occurrence of the menopausal transition. Menopause, 11, 601-606.

Steiner, A. Z., Herring, A. H., Kesner, J. S., Meadows, J. W., Stanczyk, F. Z., Hoberman, S. and Baird, D. D. (2011) Antimullerian hormone as a predictor of natural fecundability in women aged 30-42 years. Obstetrics and Gynecology, 117, 798-804.

McIlveen, M., Skull, J. D. and Ledger, W. L. (2007) Evaluation of the utility of multiple endocrine and ultrasound measures of ovarian reserve in the prediction of cycle cancellation in a high-risk IVF population. Human Reproduction, 22, 778-785.

Kwee, J., Schats, R., McDonnell, J., Themmen, A., de Jong, F. and Lambalk, C. (2008) Evaluation of anti-Mullerian hormone as a test for the prediction of ovarian reserve. Fertility and Sterility, 90, 737-743.

van Rooij, I. A. J., Broekmans, F. J. M., te Velde, E. R., Fauser, B. C. J. M., Bancsi, L. F. J. M. M., de Jong, F. H. and Themmen, A. P. N. (2002) Serum anti-Mullerian hormone levels: a novel measure of ovarian reserve. Human Reproduction, 17, 3065-3071.

Fong, S. L., Lugtenburg, P. J., Schipper, I., Themmen, A. P. N., de Jong, F. H., Sonneveld, P. and Laven, J. S. E. (2008) Anti-mullerian hormone as a marker of ovarian function in women after chemotherapy and radiotherapy for haematological malignancies. Human Reproduction, 23, 674-678.

Keizer-Schrama, S. M. P. F. D., van Beek, R. D., van den Heuvel-Eibrink, M. M., Laven, J. S. E., de Jong, F. H., Themmen, A. P. N., Hakvoort-Cammel, F. G., van den Bos, C., van den Berg, H. and Pieters, R. (2007) Anti-mullerian hormone is a sensitive serum marker for gonadal function in women treated for Hodgkin's lymphoma during childhood. J Clin Endocr Metab, 92, 3869-3874.

McDade, T. W., Williams, S. and Snodgrass, J. J. (2007) What a drop can do: dried blood spots as a minimally invasive method for integrating biomarkers into population-based research. Demography, 44, 899-925.

McDade, T. W., Burhop, J. and Dohnal, J. (2004) High-sensitivity enzyme immunoassay for C-reactive protein in dried blood spots. Clin Chem, 50, 652-654.

McDade, T. W. and Shell-Duncan, B. (2002) Whole blood collected on filter paper provides a minimally invasive method for assessing human transferrin receptor level. J Nutr, 132, 3760-3763.

McDade, T. W., Stallings, J. F., Angold, A., Costello, E. J., Burleson, M., Cacioppo, J. T., Glaser, R. and Worthman, C. M. (2000) Epstein-Barr virus antibodies in whole blood spots: a minimally invasive method for assessing an aspect of cell-mediated immunity. Psychosom Med, 62, 560-567.

Kumar, A., Kalra, B., Patel, A., McDavid, L. and Roudebush, W. E. (2010) Development of a second generation anti-Mullerian hormone (AMH) ELISA. J Immunol Methods, 362, 51-59.

Bland, J. M. and Altman, D. G. (1999) Measuring agreement in method comparison studies. Stat Methods Med Res, 8, 135-160.

Bland, J. M. and Altman, D. G. (1986) Statistical methods for assessing agreement between two methods of clinical measurement. Lancet, 1, 307-310.

Worthman, C. M. and Stallings, J. F. (1994) Measurement of gonadotropins in dried blood spots. Clinical Chemistry, 40, 448-453.

Worthman, C. M. and Stallings, J. F. (1997) Hormone measures in finger-prick blood spot samples: New field methods for reproductive endocrinology. American Journal of Physical Anthropology, 104, 1-22.

Borders, A. E. B., Grobman, W. A., Amsden, L. B., Collins, E. T. and Holl, J. L. (2007) Factors that influence the acceptability of collecting in-home finger stick blood samples in an urban, low-income population. J Health Care Poor U, 18, 100-115.

Williams, S. R. and McDade, T. W. (2009) The Use of Dried Blood Spot Sampling in the National Social Life, Health, and Aging Project. J Gerontol B-Psychol, 64, 1131-1136.

McDade, T. W. (2011) The state and future of blood-based biomarkers in the Health and Retirement Study. Forum for Health Economics and Policy, 14, article 5.

Harris, K. M. (2010) An Integrative Approach to Health. Demography, 47, 1-22. Almog, B., Shehata, F., Suissa, S., Holzer, H., Shalom-Paz, E., La Marca, A., Muttukrishna, S., Blazar, A., Hackett, R., Nelson, S. M. et al. (2011) Age-related normograms of serum antimullerian hormone levels in a population of infertile women: a multicenter study. Fertility and Sterility, 95, 2359-U2280.

Lie Fong S, Visser J A, Welt C K, de Rijke Y B, Eijkemans M J, Broekmans F J, Roes E M, Peters W H, Hokken-Koelega A C, Fauser B C, Themmen A P, de Jong F H, Schipper I, Laven J S. Serum anti-mullerian hormone levels in healthy females: a nomogram ranging from infancy to adulthood. J Clin Endocrinol Metab. 2012;97:4650-5

Freeman E W, Sammel M D, Lin H, Gracia C R. Anti-mullerian hormone as a predictor of time to menopause in late reproductive age women. J Clin Endocrinol Metab. 2012; 97:1673-80.

Kaori I, Ayako T, Kazuhiro A, Ryosuke T, Tsuyoshi H, Ippei T, Hideki M. Role of antimüllerian hormone as a biomarker of the menopausal transition. Menopause. 2012 Sep. 10. [Epub ahead of print]

Satwik R, Kochhar M, Gupta S M, Majumdar A. Anti-mullerian hormone cut-off values for predicting poor ovarian response to exogenous ovarian stimulation in in-vitro fertilization. J Hum Reprod Sci. 2012 May;5(2):206-12.

Dillon K E, Sammel M D, Prewitt M, Ginsberg J P, Walker D, Mersereau J E, Gosiengfiao Y, Gracia C R. Pretreatment antimüllerian hormone levels determine rate of posttherapy ovarian reserve recovery: acute changes in ovarian reserve during and after chemotherapy. Fertil Steril. 2012 Oct. 18. doi:pii: S0015-0282(12)02258-3. 10.1016/j.fertnstert.2012.09.039. [Epub ahead of print]

Rosenfield R L, Wroblewski K, Padmanabhan V, Littlejohn E, Mortensen M, Ehrmann D A. Antimüllerian hormone levels are independently related to ovarian hyperandrogenism and polycystic ovaries.Fertil Steril. 2012 July;98(1):242-9. doi: 10.1016/j.fertnstert.2012.03.059. Epub 2012 Apr. 26. Erratum in: Fertil Steril. 2012 September;98(3):770.

Dewailly D, Gronier H, Poncelet E, Robin G, Leroy M, Pigny P, Duhamel A, Catteau-Jonard S.Diagnosis of polycystic ovary syndrome (PCOS): revisiting the threshold values of follicle count on ultrasound and of the serum AMH level for the definition of polycystic ovaries.Hum Reprod. 2011 November;26(11):3123-9. doi: 10.1093/humrep/der297. Epub 2011 Sep. 16.

Hart R, Doherty D A, Norman R J, Franks S, Dickinson J E, Hickey M, Sloboda D M. Serum antimullerian hormone (AMH) levels are elevated in adolescent girls with polycystic ovaries and the polycystic ovarian syndrome (PCOS).Fertil Steril. 2010 Aug.;94(3):1118-21. doi: 10.1016/j.fertnstert.2009.11.002. Epub 2010 Jan. 8.

Piouka A, Farmakiotis D, Katsikis I, Macut D, Gerou S, Panidis D.Anti-Mullerian hormone levels reflect severity of PCOS but are negatively influenced by obesity: relationship with increased luteinizing hormone levels.Am J Physiol Endocrinol Metab. 2009 Feb.;296(2):E238-43. doi: 10.1152/ajpendo.90684.2008. Epub 2008 Oct. 28.

Pigny P, Jonard S, Robert Y, Dewailly D Serum anti-Mullerian hormone as a surrogate for antral follicle count for definition of the polycystic ovary syndrome. J Clin Endocrinol Metab. 2006 March;91(3):941-5. Epub 2005 Dec. 20.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of determining the approximate concentration of Anti-Mullerian hormone (AMH) in a whole blood sample comprising:
a) contacting a whole blood sample from a subject with first antibodies specific for AMH under conditions such that a signal is generated that is proportional to the approximate concentration of AMH in said whole blood sample; and
b) detecting the approximate level of said signal, thereby determining said approximate concentration of AMH in said whole blood sample.

2. The method of claim 1, wherein said first antibodies are labeled with first nanoparticles that produce a colorimetric or fluorescent signal when aggregated.

3. The method of claim 1, wherein said first nanoparticles comprise gold nanoparticles or fluorescent particles.

4. The method of claim 1, further comprising contacting said whole blood sample with a second antibodies specific for a non-AMH protein in whole blood.

5. The method of claim 1, wherein said second antibodies are labeled with first nanoparticles that produce a colorimetric or fluorescent signal when aggregated.

6. The method of claim 1, wherein said contacting is conducted on a membrane, wherein said membrane comprises: at least one test capture region which comprises third antibodies specific for said AMH or said first antibodies.

7. The method of claim 6, wherein said membrane further comprises: a control capture region which comprises fourth antibodies specific for said non-AMH protein or said second antibodies.

8. The method of claim 1, wherein said detecting said level of said signal comprises detecting the fluorescence absorbance level, the colorimetric intensity level, or the number of colorimetric symbols from, said signal.

9. The method of claim 1, further comprising comparing said approximate amount of said signal to reference signals of known AMH concentration in order to determine said approximate concentration of AMH in said whole blood sample.

10. The method of claim 1, wherein said whole blood sample has a volume of 1 drop of whole blood or less.

11. The method of claim 1, wherein said whole blood sample comprises oxygenated whole blood or a dried blood sample.

12. The method of claim 1, wherein said approximate concentration of AMH detected in said whole blood sample is greater than 3.5 ng/ml, wherein said subject is a female, and wherein said method further comprises at least one of the following steps:
i) informing said subject that she has, or likely has, polycystic ovarian syndrome;
ii) preparing and/or transmitting an electronic and/or paper report that indicates said subject has, or likely has, polycystic ovarian syndrome;
iii) preparing and/or transmitting an electronic and/or paper report that said subject should be further evaluated for polycystic ovarian syndrome;
iv) prescribing medication and/or surgical treatment to said subject to treat polycystic ovarian syndrome; and
v) treating said subject with medication or surgical treatment directed toward alleviating polycystic ovarian syndrome.

13. A lateral flow immunoassay device for detecting Anti-Mullerian hormone (AMH) in whole blood comprising:
a) a sample pad configured for receiving and transmitting a whole blood sample;
b) a conjugate pad in contact with said sample pad and configured for receiving said whole blood sample from said sample pad, wherein said conjugate pad comprises:
i) first antibodies specific for AMH, wherein said first antibodies are labeled with first nanoparticles that produce a first colorimetric or fluorescent signal when aggregated,
ii) second antibodies specific for a non-AMH protein in whole blood, wherein said second antibodies are labeled with second nanoparticles that produce a second colorimetric signal when aggregated;
c) a membrane in contact with said conjugate pad and configured to receive said whole blood sample from said conjugate pad, wherein said membrane comprises:
i) at least one test capture region which comprises third antibodies specific for said AMH or said first antibodies, and
d) a substrate, wherein said sample pad, said conjugate pad, and said membrane, are supported by said substrate.

14. The lateral flow immunoassay device of claim 13, wherein said membrane further comprises: ii) a control capture region which comprises fourth antibodies specific for said non-AMH protein or said second antibodies.

15. The lateral flow immunoassay device of claim 13, further comprising a wick component in contact with said membrane and configured to absorb excess whole blood sample.

16. The lateral flow immunoassay device of claim 13, wherein said conjugate pad, said membrane, and said wick component are attached to said substrate.

17. The lateral flow immunoassay device of claim 13, wherein said at least one test capture region comprises at least two test capture regions.

18. The lateral flow immunoassay device of claim 13, wherein said third antibodies are present in said at least one test capture region at an excess level compared to the maximum level of AMH that could be present in the amount of said whole blood that could reach said at least one test capture region.

19. The lateral flow immunoassay device of claim 13, wherein the intensity of said first colorimetric or fluorescent signal is proportional to the concentration of AMH present in said whole blood sample.

20. The lateral flow immunoassay device of claim 13, wherein said at least one test capture region is in the shape or a line, circle, or oval, and wherein said control capture region is in the shape of a line, circle, or oval.

21. The lateral flow immunoassay device of claim 13, further comprising a blood reservoir located on top of said sample pad, wherein said blood reservoir is configured to receive a dried blood sample.

22. A kit comprising:
a) said lateral flow immunoassay device of claim 13, and
b) at least one component selected from the group consisting of:
i) at least one sterile lancet,
ii) a gas impermeable foil bag,
iii) a color chart, wherein said color chart allows a user of said lateral flow immunoassay to estimate the concentration of AMH in a whole blood sample tested on said lateral flow immunoassay device by comparison to said color chart,
iv) a sterile gauze pad,
v) a skin sterilization wipe,
vi) printed instructions for collecting blood and applying it to said lateral flow immunoassay device,
vii) printed instructions for interpreting said first colorimetric signal,
viii) a piece of filter paper for collecting a dried blood sample, and
ix) a container for housing said lateral flow immunoassay device.

23. A method of using a lateral flow immunoassay device for detecting Anti-Mullerian hormone (AMH) in a whole blood sample comprising:
applying a whole blood sample from a subject to said sample pad of said lateral flow immunoassay device of claim 13 under conditions such that at least a portion of said whole blood sample migrates from said sample pad, through said conjugate pad to said at least one test capture region and said control capture region in said membrane thereby generating said first and second colorimetric or fluorescent signals,
wherein said first colorimetric/fluorescent signal is proportional to the approximate concentration of AMH in said whole blood sample.

24. The method of claim 23, wherein said approximate concentration of AMH detected in said whole blood sample is greater than 3.5 ng/ml, wherein said subject is a female, and wherein said method further comprises at least one of the following steps:
i) informing said subject that she has, or likely has, polycystic ovarian syndrome;
ii) preparing and/or transmitting an electronic and/or paper report that indicates said subject has, or likely has, polycystic ovarian syndrome;
iii) preparing and/or transmitting an electronic and/or paper report that said subject should be further evaluated for polycystic ovarian syndrome;
iv) prescribing medication and/or surgical treatment to said subject to treat polycystic ovarian syndrome; and
v) treating said subject with medication or surgical treatment directed toward alleviating polycystic ovarian syndrome.
